(19) United States
(12) Patent Application Publication
(10) Pub. No.: US 2010/0196278 A1
(43) Pub. Date: Aug. 5, 2010

(54) PHOTOACOUSTIC IMAGING AGENT

(75) Inventor: Yoshinori Tomida, Atsugi-shi (JP)

Correspondence Address: FITZPATRICK CELLA HARPER & SCINTO, 1290 Avenue of the Americas, NEW YORK, NY, 10104-3800, US

(73) Assignee: Canon Kabushiki Kaisha, Tokyo (JP)

(21) Appl. No.: 12/678,348

(22) PCT Filed: Nov. 14, 2008
(86) PCT No.: PCT/JP2008/071169

§ 371 Date: Mar. 16, 2010

(30) Foreign Application Priority Data
Nov. 16, 2007 (JP) 2007-298214

(51) Int. Cl. A61K 49/00

(52) U.S. Cl. 424/9.1

(57) ABSTRACT

A molecular probe marked with a color center material is used as a photoacoustic imaging agent to obtain an acoustic signal of practically adequate intensity using weak near-infrared light, which has good in vivo penetration depth but has small excitation energy, and is within the maximum permissible exposure, in photoacoustic tomography (PAT) diagnosis of a living body.

### TECHNICAL FIELD

The present invention relates to a photoacoustic imaging agent for use in photoacoustic tomography (PAT) diagnosis.

### BACKGROUND ART

Increase in the incidence of diseases like tumors and arteriosclerosis has become a major social problem.

Invasive methods that use in vivo imaging apparatuses, such as x-ray CT and PET-CT, have been in use for diagnosing such diseases, but there is a demand for less invasive methods. Photoacoustic tomography is a candidate for such less invasive methods. Photoacoustic tomography itself (the light irradiation part itself) is a noninvasive modality. However, the difference in optical properties between the normal and the diseased parts, such as those having tumors, arteriosclerosis, and the like, is not sufficiently large to be detected by photoacoustic tomography. Therefore, an imaging agent needs to be administered while using photoacoustic tomography to enhance the contrast, for diagnosing tumors and arteriosclerosis. In short, photoacoustic tomography diagnosis may be considered a minimally invasive method. FIG. 2 is a schematic diagram of a conventional in vivo imaging apparatus. A water tank 5 is filled with water and the object of interest 6 in the water is irradiated with light 16 from a light source 7. The light 16 from the light source 7 is irradiated via an optical system, such as a mirror 8 and a concave lens 9. Pulsed acoustic signals emitted from the object of interest when it is irradiated with the light is then gathered by a transducer 10 through the water, and the signal obtained by scanning while rotating the transducer, keeping its center of rotation at about the center of the sample (the object of interest), is then image-processed to obtain an in vivo image. 11 is an amplifier, 12 is an oscilloscope, 13 is a computer, 14 is a step motor and 15 is an oscillator element. The light source for the photoacoustic imaging is near-infrared light in the range 600 nm to 1300 nm, which has high in vivo penetration depth. “Noninvasive photoacoustic angiography of animal brains in vivo with near-infrared light and an optical contrast agent” by Xueding Wang, et al., Optics Letters Vol. 29, No. 7, pp. 730-732, Apr. 1, 2004, may be referred to for information about conventional photoacoustic imaging.

In Xueding Wang, et al., Optics Letters Vol. 29, No. 7, pp. 730-732, Apr. 1, 2004, indocyanine green (ICG) and polyethylene glycol-stabilized indocyanine green (ICG-PEG) are injected intravenously into rats as contrast imaging agents and the cranial blood vessels are imaged through photoacoustic imaging. The absorption spectra of the contrast imaging agents used are given in the document. The laser beam intensity that can be used in vivo irradiation is not more than the maximum permissible exposure (MPE) specified in JIS C 6802:2005, and, as near-infrared light gets scattered in the living body, the light intensity becomes weak in the deeper regions of the body. The energy, which is the product of the intensity of the light that reaches the imaging agent and the absorption coefficient, is absorbed by the imaging agent, a part of this energy induces molecular vibrations of the imaging agent, and these vibrations are converted into heat. The heat is retained within the imaging agent, while a part of it is transmitted to the surrounding biological materials. Then they undergo micro-deformations, depending on their thermal expansion coefficients, which accordingly generates sound. This acoustic pressure is detected as the photoacoustic signal.

The conventional imaging agents described above have the following problems.

Firstly, because the light intensity in the deeper regions of the living body is low, the heat generated by the imaging agent is only of the order of several mK. In the above document (Xueding Wang, et al., Optics Letters Vol. 29, pp. 730-732, 2004), this was 2.6 mK for an exposure intensity of 2 mJ/cm². Therefore, in the deeper regions of the body, the acoustic pressure, which is to be detected, as the photoacoustic signal is invariably small, and the contrast of the images obtained of the deep regions of body is very low.

Secondly, near-infrared light has low energy compared to wavelengths of the UV, therefore the energy of near-infrared light or the energy of heat converted from near-infrared light cannot promote reactions. These energies can cause molecular rotation and molecular vibration, but they cannot sever molecular bonds because they are smaller than the bond energy of atoms that constitute the molecules or the activation energy of chemical reactions.

Thirdly, in case that a dye is used as imaging contrast agent, an additive effect can be achieved at most up to about 10¹⁷ per cm³. This is because when the dye concentration is increased, phenomena like concentration quenching, and increased optical anisotropy in the aggregated state, which causes anisotropy in light absorption, polarization of light absorption, shift in the light absorption wavelengths, and saturation and lowering of light absorption efficiency occur.

On the other hand, some color center materials are known. Color center materials are colorless, transparent, monocrystals. But when they are irradiated with a primary excitation light, like γ-rays, x-rays, electron beam, or UV light, or exposed to alkali metal vapors, they get colored and start absorbing light of specific wavelength bands. Light absorption spectra of color center materials are given in Henry F. Ivey, “Spectral Location of the Absorption Due to Color Centers in Alkali Halide Crystals”, Physical Review, Vol. 72, No. 4, pp. 341-343, Aug. 15, 1947.

### DISCLOSURE OF THE INVENTION

To solve the aforementioned problems, the present invention aims at providing a photoacoustic imaging agent having, as its light absorbing component, particles with a structure that can convert an input signal, which is the faint light that arrives when near-infrared light with intensity not more than MPE is dispersed within the body, into a large acoustic output signal.

The photoacoustic imaging agent of the present invention, which aims at solving the aforementioned problems, is for use in photoacoustic tomography (PAT) diagnosis, comprises a particle as a light absorbing component, wherein the particle comprises:

a core part that includes crystals comprising one of an alkali halide and an alkali earth halide, which can form color centers that absorb light in the wavelength band used for the PAT diagnosis;

a shell part that covers the core part to prevent the core part from coming into contact with the external environment; and

a marker part that selectively reacts with a specific disease to be detected,

wherein the overall mean particle size, of the core part, shell part, and the marker part combined, being 100 nm or less.

### BRIEF DESCRIPTION OF THE DRAWINGS

FIGS. 1A and 1B are schematic diagrams of the photoacoustic contrast imaging agent of the present invention.

FIG. 2 is a diagram illustrating a conventional photoacoustic measurement modality.

FIGS. 3A and 3B are diagrams describing the mechanism of formation of color centers used in the photoacoustic imaging agent of the present invention.

FIG. 4 illustrates measured absorption spectra of various ionic crystals that formed color centers.

FIG. 5 illustrates measured absorption spectra of various ionic crystals that formed color centers.

FIG. 6 illustrates the photoacoustic signal produced when the color centers of sodium bromide crystals were irradiated with 532 nm light.

FIG. 7 is diagram of different types of color centers.

### BEST MODES FOR CARRYING OUT THE INVENTION

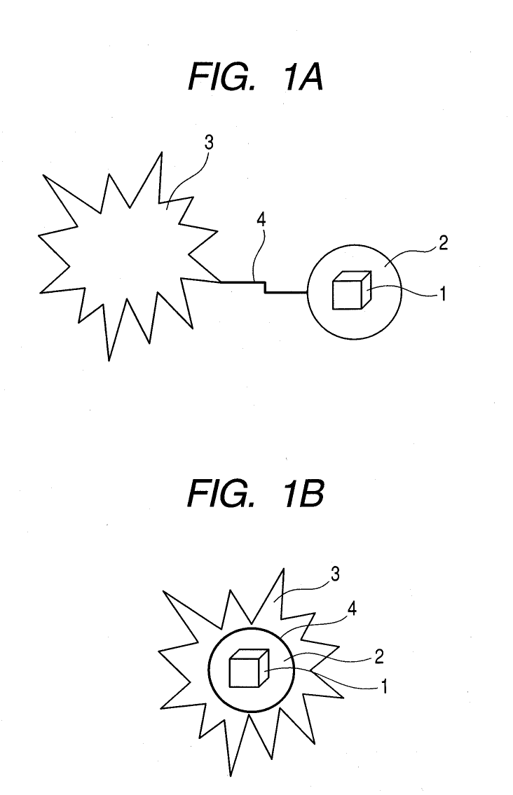

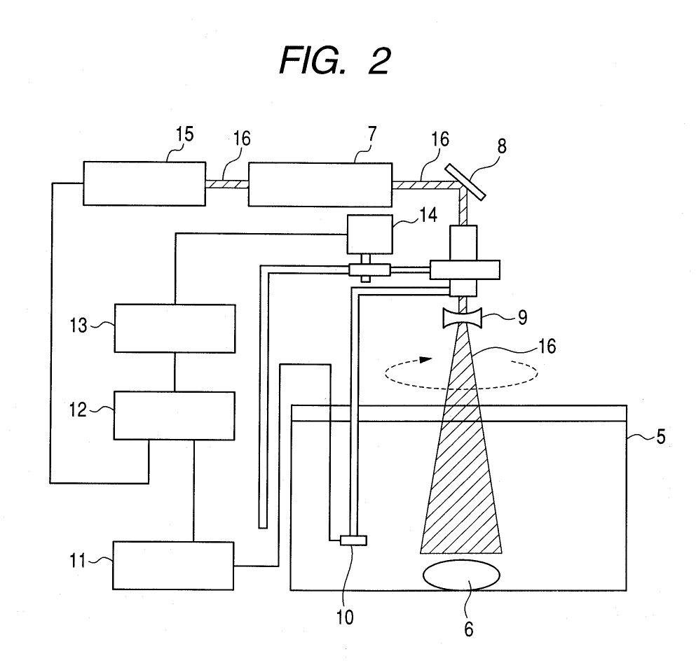

The photoacoustic imaging agent of the present invention will be described in greater detail below. FIGS. 1A and 1B are schematic diagrams of examples of the structure of photoacoustic imaging agents of the present invention. The effective component, i.e., the light-absorbing component particle, of the photoacoustic imaging agent includes one of crystals comprising an alkali halide and crystals comprising an alkali earth halide (the crystals can have both alkali halide and alkali earth halide) as a core part 1. The core part forms color centers when irradiated with the primary excitation light or when exposed to alkali metal vapor. A shell part 2 is provided as an outer shell that covers the core part 1. Furthermore, a marker part 3 is provided on the outer surface of the shell part 2. A conjugating part 4 that connects the shell part 2 and marker part 3 is provided, when required, between these two parts. FIG. 1A illustrates an example where the conjugating part 4 connects the shell part 2 and the marker part 3, which is a separate entity. FIG. 1B is a modified example where the shell part 2 is within the marker part 3, or the marker part 3 includes the shell part 2.

(Materials that can Form Color Centers and Methods of Creating Color Centers)

The alkali halide or alkali earth halide used in the photoacoustic agent of the present invention is a salt comprising a combination of one or more cations selected from among lithium, sodium, potassium, rubidium, cesium, francium, beryllium, magnesium, calcium, strontium, barium and radium, and one or more anions selected from fluorine, chlorine, bromine, iodine, and astatine. A mixture of two or more types of salts can be used. Complex salts comprising a plurality of these cations and a plurality of these anions can also be used. Furthermore, perhalogenates formed by combining such as perchloric acid anions or perbromic acid anions with an alkali cation or alkali earth cation can also be used. In the present invention, the perhalogenate is considered as a halide.

The aforementioned substances generally do not have optical absorption in the wide wavelength range from the visible wave length region to the infrared region, and therefore, their monocrystals are normally colorless and transparent. On the other hand, when the aforementioned substances are irradiated with a primary excitation light like γ-rays, x-rays, electron beam, and UV light, or exposed to alkali metal vapors, they get colored and start absorbing light of specific wavelength bands. Hereinafter, materials with this property are collectively referred to as “color center materials”.

(Basic Property of Color Center Materials)

Color centers have been described in the references listed below, which are in the public domain.
Hikari Bussei Handobukku (Handbook of Optical Properties)” (p. 228, p. 398)
“Color Centers in Alkali-Halides, and Allied Phenomena” (Electronic Processes in Ionic Crystals, p. 109)
“Hikari Bussei, Denshi Koshi Sogosayo (Optical Properties and Electron-Lattice Interactions)” (Supplementary Volume of Kotai Butsuri (Solid Physics), p. 17, p. 82)
“Busshitsu to Hikari (Materials and Light)” (Rikogaku Kiso Koza 24, p. 130)

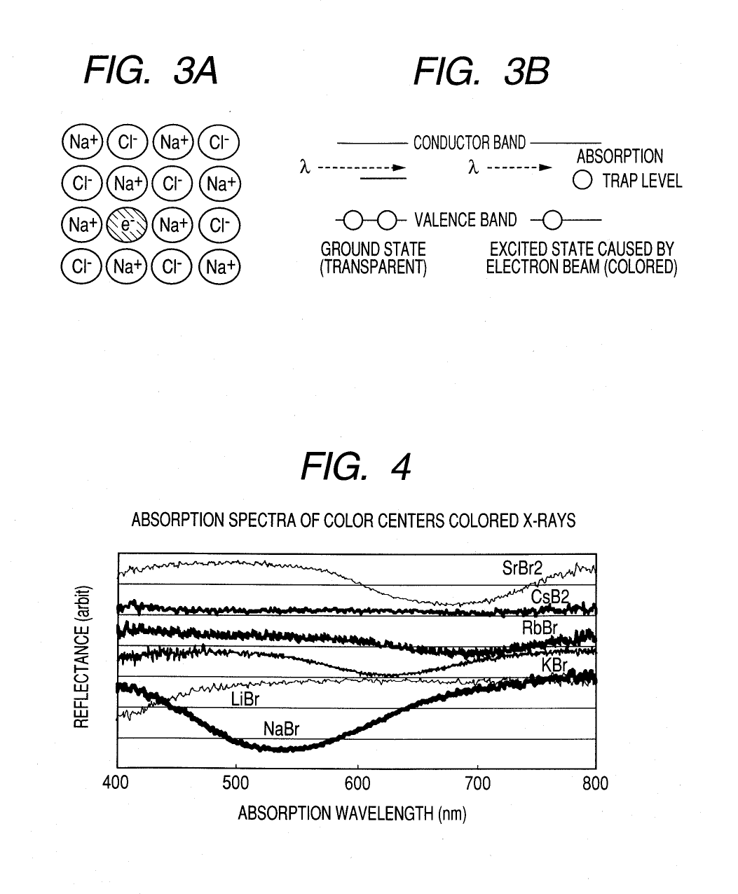

FIG. 3A and FIG. 3B are diagrams that illustrate the principle of coloration of color center materials, structurally and energetically.

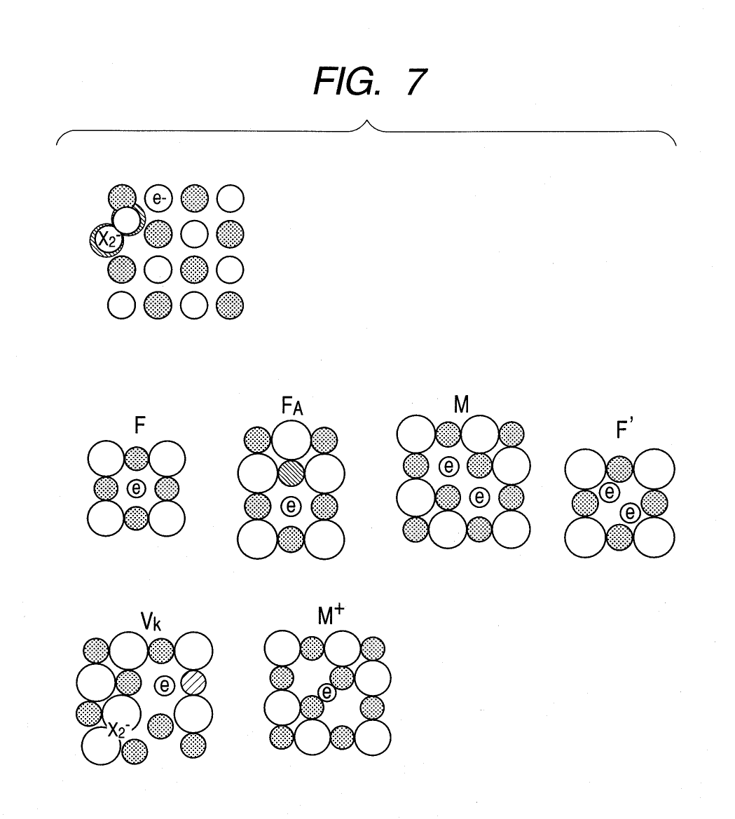

FIG. 7 is a diagram that illustrates different types of color centers. In the figures, the “circles marked as e or e⁻” are electrons. “Dotted circles”, “hatched circles”, and “white circles” respectively represent cations, holes, and anions. Color centers do not always maintain only one structure among the structures illustrated in FIG. 7; they are known to undergo conversions (changes in type). The color centers of this invention can be of any of these types as long as it has absorbance at the wavelength used for the photoacoustic imaging in the present invention. Apart from the condition where the electrons are localized as illustrated in FIG. 7, a hydrogen atom-like model where the electron is not actually localized has also been suggested. Such color centers can also be used in the present invention. That color centers sometimes assume a hydrogen atom-like structure is said to be the reason why they have high oscillator strength, in other words, high optical absorption efficiency.

The mechanism of coloration is understood to be the trapping of an exciton comprising an electron-hole pair within the ionic crystal as illustrated in FIG. 3A and FIG. 7.

When the primary excitation light is irradiated, the exciton gets trapped inside the crystal in the form an “electron−hole” pair. The “hole” cannot exist independently for a long time, but a stabilized model (Vk) is conceivable. In the Vk model, a “hole (valency +1)” gets trapped in the “halogen anion pair (valency−2)” created because of transposition of electron coordinates of the two “halogen anions (example: assumed valency −1)” by the impact of the excitation, forming a “halogen anion pair+hole (valency−1)”, and stabilizing the hole. The development of color when irradiated with the primary excitation light may be described as follows. In short, energetically, a trap level (of halogen) exists between the conductor and the valence band, as illustrated in FIG. 3B. The primary excitation light excites the electron, which moves to the trap level from the valence band. Hereinafter, this is referred to as primary excitation. The state where the electron is excited to the trap level is referred to as the excited state. A characteristic feature of this excited state is that its lifetime is relatively long at around room temperature.

On the other hand, coloration when exposed to alkali metal vapors is said to be caused by trapping of the alkali metal in the form of an “alkali ion (alkali cation)−electron” pair. In this case, it is believed that the exciton comprising an “electron-alkali cation pair” gets trapped at a lattice point where an “alkali cation” and a “halogen anion” are lacking in the Schottky crystal, i.e., the original crystal. The electron is in the excited state in this case also.

In both the cases described above, the color center assumes an excited state.

If a color center in this excited state is irradiated with light of its absorption wavelength band, the trapped electron can be photo-excited from its trap level to the conduction band. (Hereinafter, this is referred to as secondary excitation). A large amount of energy corresponding to the primary excitation is released in the process where the electron, which has been excited to the conduction band by secondary excitation, returns to its ground state in the absence of radiation. In short, a color center can release a large amount of energy, corresponding to the primary excitation light, in response to the small energy of the secondary excitation light. FIG. 3A illustrates an NaCl type crystal lattice. But similar trapping of excitons occurs and color centers are created in CsCl type crystal lattices also.

(Correlation Between the Absorption Wavelength of the Color Center and Chemical Composition of the Core Part)

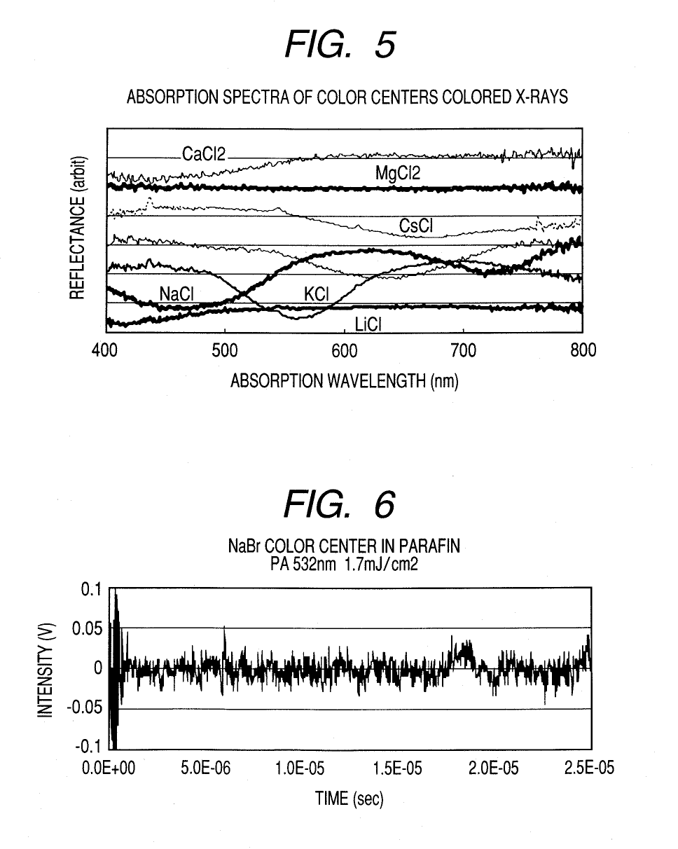

The light absorption band of the color centers formed in the crystals used in the core part can be adjusted by combining different ionic species from a group of materials that can form color centers. In other words, color centers that absorb in the wavelength band of the light used for photoacoustic imaging, can be created in the crystals used in the core part by selecting a combination of ionic species of materials capable of forming color centers. Such selections can be made, for instance, based on the absorption spectra, of the type illustrated in FIGS. 4 and 5, of color centers formed when bromide salts and chloride salts are irradiated with x-rays. Such selection can also be made using absorption spectra of the bromide, chloride, fluoride, and iodide salts described in the earlier-mentioned Henry F. Ivey, Physical Review, Vol. 72, pp. 341-343, 1947, etc. Examples of materials for forming the core include NaF, NaCl, NaBr, and NaI.

By using mixed crystals in which these ionic species are mixed, the absorption spectrum can be continuously varied by changing to the mixing ratio. Therefore, color centers with suitable absorption spectra can be designed. The present invention is thus characterized by the fact that mixed crystal composition is made in such a way that its color center absorption spectrum matches with the wavelength band of the light used in photoacoustic imaging. For example, salts in which 3 or 4 components, like one or more of cations (A and B for instance) and one or more of anions (C and D for instance), are “mixed” (for instance, salts represented by the compositional formulas A_{0.5}B_{0.5}C, A_{0.5}B_{0.5}C_{0.5}D_{0.5}, and A_{0.2}B_{0.8}C_{0.7}D_{0.3}) can be used.

Moreover, absorption spectral peaks of the above-mentioned color centers (absorption spectral peaks during the secondary excitation), have narrower wavelength bands compared to the broad near-infrared spectral peak of ICG described in the conventional example, and therefore, give high effective quantum efficiency when a laser is used.

Near-infrared light of wavelength 600 nm to 1300 nm inclusive can be used for the secondary excitation while using the photoacoustic imaging agent of the present invention. The optical absorption by blood hemoglobin and by water is low in this wavelength band, which is also known as the “optical window”, and therefore it is currently being studied for use as a light source for in vivo irradiation of light.

As the present invention relates to an imaging agent, the absorption wavelength of the color center can be adjusted to correspond to the wavelength of the light used in photoacoustic tomography equipment, i.e., the light irradiated on the imaging agent.

A tunable dye laser, which is expensive, is used for light irradiation of the dye described in the conventional example. Contrary to this, as the absorption wavelength of the imaging agent of the present invention can be adjusted, an inexpensive general-purpose gas laser or laser diode can be used for the irradiation. The absorption peak of the color centers of the core part in the present invention can be easily adjusted to the wavelength of the laser. For example, when potassium bromide is used in the core part, the absorption peak of the color centers can be adjusted to 633 nm, which is the wavelength of helium-neon laser. Another example is the use of mixed crystals of potassium bromide and rubidium bromide at the molar ratio of 1:9 wherein the absorption peak of the color center can be adjusted to 680 nm, which is the wavelength of laser diodes developed for optical discs.

(Shell Part)

If, in the present invention, the alkali halide (or alkali earth halide) in the core part, absorbs water and deliquesces, it cannot retain its crystal structure. As is clear from its principle, the color center can exist only in a substance in a crystalline state and not in a substance in a state of solution. Therefore, providing a shell part that protects the core part from water vapor in the atmosphere and moisture in the living body is an important feature in using color centers as a contrast imaging agent. Materials that form an outer shell over the core part to prevent contact between the core part and the external environment, and can prevent the penetration of at least moisture and water vapor, are used as materials for making the shell part. This type of shell part can be formed, for instance, by coating the outer surface of the core part or by modifying the outer surface of the core part. Both organic and inorganic high molecular compounds are suitable as materials for such coating or modification. Examples of organic high molecular compounds include various polymers and polypeptides, derivatives thereof, copolymers of two or more of these, and organic dendrimers. Mixtures of two or more of these materials can also be used. Examples of polymers include polyethylene glycol, polyvinyl alcohol, polylactic acid, poly(meth)acrylic acid and its esters, polyethylene, polystyrene, polyethylene terephthalate, gelatin, silicones, and polysiloxane. Examples of inorganic high molecular compounds include silica and alumina. A complex having any one of the above-mentioned substances as the chief component can also be used. The shell material is not limited to high molecular compounds; low molecular lipid bilayers and the like can also be used as the shell.

(Marker Part and Marker-Shell Conjugate)

The marker part has a configuration that enables selective reaction with a specific disease to be detected. Markers selectively detect various diseases, such as a tumor or arteriosclerosis can be suitably used in the marker part. Such markers generally show specific and selective adsorption. An antibody that reacts selectively with an antigen, an enzyme that reacts selectively with a substrate, and a substance that has a molecular structure that causes a specific adsorption reaction with the subject to be detected in a hypoxic region or a low pH region, etc can be used as the marker.

To conjugate the marker part and the shell part, for instance, a molecule having a terminal carboxyl group can be used for the above-mentioned shell part, and the technique of converting the carboxyl group into a succinimide ester, and then forming an amide bond with an amino group of the antibody or enzyme, can be used. An example of another method of conjugate formation is to convert the ends of the molecules that constitute the shell part into maleimides and then to condense them with thiol groups of the antibody or enzyme.

(Particle Size)

The number of excitons trapped inside the crystal, i.e., the number of color centers formed, as a result of the primary excitation depends on the wavelength and output of, and the duration of exposure to, the primary excitation light, and also the defect density and impurity concentration, etc of the crystals. Its upper limit is said to be 10¹⁸ per cm³. When the density of color centers is 10¹⁸ per cm³, according to calculations there will be 1000 color centers in a 100 nm cube.

Crystals of nanoparticle size in the range of several 10s of nm to 100 nm can be obtained from aqueous solutions of alkali halide (or alkali earth halide) by the spray-drying method or inkjet method. Spray-drying is a method in which an aqueous solution of an alkali halide is sprayed and the fine liquid droplets formed are exposed to dry air to crystallize them as nanoparticles. In the inkjet method, fine droplets extruded from a fine nozzle are dried or dropped into a poor solvent of the alkali halide, such as butanol, to obtain fine crystals.

The whole particle, after the shell is formed outside the core, and the marker part is conjugated to make it into the final imaging agent, can be of a size that enables its use in drug delivery in vivo. Considering the vascular permeability, the particle size (the size of the entire particle, which is the combination of the core, the shell, and the marker parts: hereinafter this will be discussed on the basis of the mean particle size) can be 100 nm or less, the preferable range being 50 nm to 100 nm. The risk of causing thrombosis when administered into the living body can be minimized by keeping the mean particle size not more than 100 nm. Apart from the particle size, whether the particles are hydrophilic or hydrophobic, and the higher order structure of the molecule, also contribute in determining the vascular permeability of the particles. Therefore, a particle size of not more than 100 nm is a rough guideline. Here, when the particles are not spherical, the mean value of the largest diameter of each particle of the entire population of particles is taken as the mean particle size.

(Timing of Color Center Formation)

The particles, which are the effective light absorbing components of the imaging agent of the present invention, can be stored and transported in a state where the core part, shell part and marker part are bonded together. The imaging agent of the present invention can be prepared from these particles, which are the effective light absorbing components, alone, or by mixing them with carriers and a diluent, if required. When using the imaging agent under the condition where the color centers are not yet formed, the core part of the imaging agent is subjected to primary excitation at the preparatory stage of acquiring the image by photoacoustic tomography (PAT), and then the agent is administered to the subject to be diagnozed. In that case, coloration occurs and color centers having light absorption in the wavelength band of the light used for PAT diagnosis are formed in the core part when irradiated with a primary excitation light, like γ-rays, x-rays, electron beam, and UV light, as described earlier. The shell part and the marker part are almost transparent to the primary excitation light, and therefore, the excitation light is absorbed mainly by the core part.

### Examples

The present invention is described in detail below, using examples.

### Example 1

Preparation of Photoacoustic Imaging Agent

0.5 g of potassium bromide (molecular weight 119.01) was dissolved in pure water and the volume made up to 1 ml to prepare 4.2 M aqueous solution (this concentration was close to the saturation concentration of 1 g/1.5 ml). This aqueous solution of potassium bromide was crushed and dispersed with pressurized air and the mist produced was graded and mixed with dry air of normal temperature to prepare nanocrystals of size 60 nm to 100 nm. AP-9000G manufactured by Shibata Scientific Technology Ltd. was used as the particle generator in this step. The mean particle size was measured by the dynamic light scattering method.

These potassium bromide fine crystals were dispersed, as the core part, in paraffin, and polyethylene glycol having terminal N-hydroxysuccinimide ester (NHS) and mean molecular weight 12,000 (manufactured by NOF Corporation) was added thereto to adsorb it around the core part to form a core-shell structure. The core-shell structure thus obtained was extracted into an aqueous phase to obtain a dispersion in water. After that, the shell part was labeled with an antibody as a marker part against a receptor that is expressed in mouse macrophage, thus conjugating the marker part. The mean particle size of these particles, as determined with a laser diffraction particle size distribution analyzer, was 70 nm to 100 nm. The marker-conjugates were then irradiated with x-rays (Cu—Kα beam, 45 kV 40 mA) for more than 1 hour, and it was confirmed that color centers had formed, as the potassium bromide turned blue. This material could be used as a photoacoustic imaging agent.

### Example 2

Diagnosis using the Photoacoustic Imaging Agent

The material in Example 1 is used as a photoacoustic imaging agent. The entire amount of the agent was intravenously administered into a mouse that expressed the mouse macrophage receptor in its lungs. 30 minutes later, 633 nm He—Ne laser pulses were irradiated at the intensity of 32 mJ/cm², and the acoustic signals from the receptor-expressing part of the mouse's lung were measured in water with a plurality of Immersion Transducers (proprietary name, manufactured by Toray Engineering Co. Ltd.). During this procedure, at least the part of the mouse that contained the lungs was positioned in water. The distance between the receptor-expressing site and the Immersion Transducers was estimated from the time delay between the irradiation of the laser pulses and the time at which the acoustic signals were detected, as illustrated in FIG. 6. The acoustic signals observed at several points around the mouse were image-processed by the coded block pattern method and the like to obtain tomograms. Acoustic signals were not observed when no photoacoustic imaging agent was administered, even when the same type of light was irradiated. It could thus be verified that the image was created due to specific accumulation of the photoacoustic imaging agent at the receptor-expressing site in the lungs.

### Example 3

Preparation of a Photoacoustic Imaging Agent having Peak Absorption at 680 nm

1 ml of a mixed aqueous solution containing 0.06 g of potassium bromide (molecular weight 119.01) and 0.74 g of rubidium bromide (molecular weight 165.39) was prepared. The potassium bromide and rubidium bromide were present in this mixed aqueous solution at an approximate molar ratio of 1:9. Formation of the nanoparticle core part, shell part, and marker conjugate was carried out using the same techniques as in Example 1 to prepare a photoacoustic imaging agent suited for near-infrared laser diode of wavelength 680 nm.

According to the preferred embodiments of the present invention described above, we can obtain photoacoustic imaging agents having a structure that can convert an input signal that is near-infrared faint light of not more than MPE into a large acoustic output signal.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2007-298214, filed Nov. 16, 2007, which is hereby incorporated by reference herein in its entirety.

1. A photoacoustic imaging agent for use in photoacoustic tomography (PAT) diagnosis comprising a particle as a light absorbing component, wherein the particle comprises:
a core part that includes crystals comprising one of an alkali halide and an alkali earth halide, which can form color centers that absorb light in the wavelength band used for the PAT diagnosis;
a shell part that covers the core part to prevent the core part from coming into contact with the external environment; and
a marker part that selectively reacts with a specific disease to be detected,
wherein the overall mean particle size, of the core part, shell part, and the marker part combined, being 100 nm or less.

2. The photoacoustic imaging agent according to claim 1 wherein the wavelength band of the light used in the PAT diagnosis is in the near-infrared region of 600 nm to 1300 nm inclusive.

3. The photoacoustic imaging agent according to claim 1 wherein the shell part includes an inorganic high molecular compound.

4. The photoacoustic imaging agent according to claim 3 wherein the inorganic high molecular compound is one of silica, alumina, and a complex with these substances as the main components.

5. The photoacoustic imaging agent according to claim 1 wherein the shell part includes an organic high molecular compound.

6. The photoacoustic imaging agent according to claim 5 wherein the shell part includes one of polyethylene glycol, polypeptide, and a derivative thereof; a copolymer of two or more of these compounds; an organic dendrimer; and a mixture of two or more thereof, as the organic high molecular compound.

7. The photoacoustic imaging agent according to claim 1 wherein the marker part includes one of an antibody and a molecular structure that causes a specific adsorption reaction in a hypoxic region or a low pH region.
